(19) United States
(12) Patent Application Publication
(10) Pub. No.: US 2019/0290873 A1
(43) Pub. Date: Sep. 26, 2019

(54) AIRWAY ACCESS ASSIST CLIP

(71) Applicant: Cook Medical Technologies LLC, Bloomington, IN (US)

(72) Inventor: James W. Willett, Ashland, VA (US)

(73) Assignee: Cook Medical Technologies LLC, Bloomington, IN (US)

(21) Appl. No.: 15/934,388

(22) Filed: Mar. 23, 2018

(51) Int. Cl. A61M 16/04

(52) U.S. Cl. CPC A61M 16/0497; A61M 16/0465; A61M 16/049; A61M 16/0402

(57) ABSTRACT

A clip for use with an airway tube assembly is provided, the clip including a body, a proximal element, and a distal element. The body extends from a proximal end to a distal end. The proximal element projects outwardly from the proximal end of the body. The proximal element is adapted to extend over a proximal end of the airway tube assembly and to restrict proximal movement of the airway tube assembly relative to the clip. The distal element projects outwardly from the distal end of the body. The distal element is adapted to be coupled to the airway tube assembly and to restrict distal movement of the airway clip relative to the clip.

### TECHNICAL FIELD

This disclosure relates to devices for use in accessing airways of patients during medical procedures, and, in particular to accessories for use with dilators and catheters.

### BACKGROUND

When accessing an airway of a patient, frequently a dilator is positioned inside an airway tube to ease insertion of the airway tube. Frequently during insertion the dilator may become separated from the airway tube making airway insertion more difficult and time-consuming. Particularly in critical care operations such as during a cricothyrotomy, a delay in insertion of the airway tube can harm the patient. For these reasons, a device to allow easy and reliable insertion of an airway tube into the airway of a patient is desirable.

### SUMMARY

Further areas of applicability will become apparent from the description provided herein. It should be understood that the description and specific examples are intended for purposes of illustration only and are not intended to limit the scope of the present disclosure.

In one embodiment, a clip for use with an airway tube assembly is provided, the clip including a body, a proximal element, and a distal element. The body extends from a proximal end to a distal end. The proximal element projects outwardly from the proximal end of the body. The proximal element is adapted to extend over a proximal end of the airway tube assembly and to restrict proximal movement of the airway tube assembly relative to the clip. The distal element projects outwardly from the distal end of the body. The distal element is adapted to be coupled to the airway tube assembly and to restrict distal movement of the airway clip relative to the clip.

In another embodiment, an airway access system is provided including an airway tube, a dilator, and a clip. The airway tube includes an outer surface and an inner surface defining a lumen. The dilator includes a proximal end and is adapted to be placed within the lumen of the airway tube. The clip includes a body, a proximal element, and a distal element. The body extends from a proximal end to a distal end. The proximal element projects outwardly from the proximal end of the body. The proximal element is adapted to extend over a proximal end of the airway tube assembly and to restrict proximal movement of the airway tube assembly relative to the clip. The distal element projects outwardly from the distal end of the body. The distal element is adapted to be coupled to the airway tube assembly and to restrict distal movement of the airway clip relative to the clip.

In yet another embodiment, a method of inserting an airway tube into a patient is provided including inserting an airway tube assembly into the patient, uncoupling a clip from the airway tube, and retracting the dilator from the lumen of the airway tube. The airway tube assembly includes the airway tube, a dilator, and a clip. The airway tube has a an inner surface which defines a lumen. The dilator is disposed within the lumen of the airway tube. The clip includes a body extending from a proximal end to a distal end. The body includes a proximal element projecting outwardly from the proximal end of the body and a distal element projecting outwardly from the distal end of the body. The proximal element is coupled to the proximal end of the dilator to restrict proximal movement of the dilator relative to the clip. The distal element is coupled to the airway tube to restrict distal movement of the airway tube relative to the clip.

Advantages of the present disclosure will become more apparent to those skilled in the art from the following description of the preferred embodiments of the disclosure that have been shown and described by way of illustration. As will be realized, the disclosed subject matter is capable of other and different embodiments, and its details are capable of modification in various respects. Accordingly, the drawings and description are to be regarded as illustrative in nature and not as restrictive.

### BRIEF DESCRIPTION OF THE DRAWINGS

The embodiments may be better understood with reference to the following drawings and description. The components in the figures are not necessarily to scale. Moreover, in the figures, like-referenced numerals designate corresponding parts throughout the different views.

FIG. 1 illustrates a perspective view of an example of an airway access system including an airway tube, a dilator, a wire guide, and a clip;

FIG. 2 illustrates a cross-section side view of a first example of the clip;

FIG. 3 illustrates a top down plan view of a first example of a proximal element of the clip;

FIG. 4 illustrates a top down plan view of a second example of a proximal element of the clip;

FIG. 5 illustrates a top down plan view of a first example of a distal element of the clip;

FIG. 6 illustrates a top down plan view of a second example of a distal element of the clip;

FIG. 7 illustrates a partial cross-sectional view of an example of the dilator and the clip;

FIG. 8 illustrates a cross-sectional side view of a first example of the airway tube;

FIG. 9 illustrates a cross-sectional side view of a second example of the airway tube;

FIG. 10 illustrates a cross-sectional side view of a second example of the clip; and

FIG. 11 illustrates a flow diagram of an example of a method of inserting an airway tube into a patient.

### DETAILED DESCRIPTION

The following description is merely exemplary in nature and is not intended to limit the present disclosure, application, or uses.

By way of an introductory example, a clip for use with an airway tube assembly is provided, the clip including a body, a proximal element, and a distal element. The body extends from a proximal end to a distal end. The proximal element projects outwardly from the proximal end of the body. The proximal element is adapted to extend over a proximal end of the airway tube assembly and to restrict proximal movement of the airway tube assembly relative to the clip. The distal element projects outwardly from the distal end of the body. The distal element is adapted to be coupled to the airway tube assembly and to restrict distal movement of the airway clip relative to the clip.

One interesting feature of the systems and methods described below may be that the clip may help operators insert the airway tube into the airway of the patient quickly and easily, preventing separation between the dilator and the airway tube. Alternatively, or in addition, an interesting feature of the systems and methods described below may be that the clip may be rapidly and easily separated from the airway tube, allowing rapid retraction of the dilator after the airway tube has been placed in the airway of the patient.

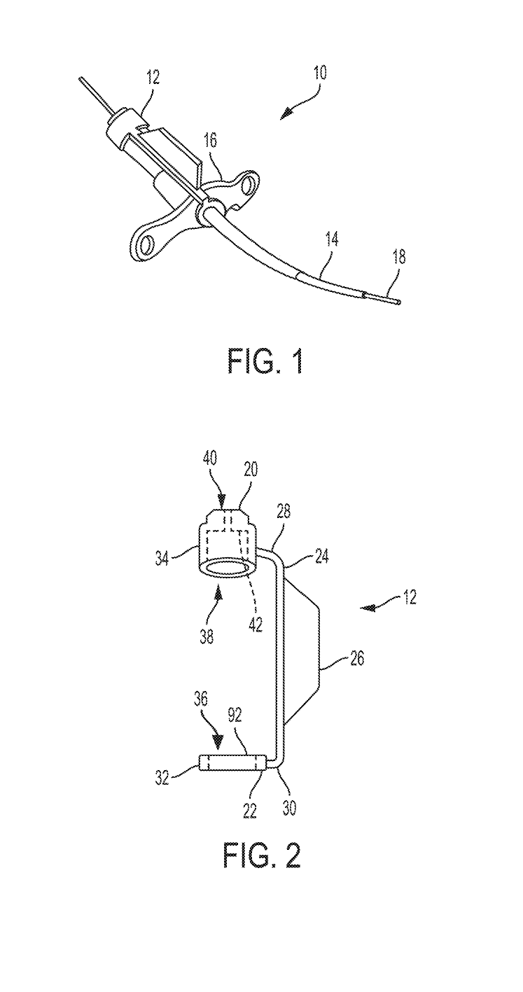

FIG. 1 illustrates an example of an airway tube assembly 10. The airway tube assembly 10 may be any device which is capable of being introduced into the airway of a patient and providing an alternative airway for the patient. Examples of the airway tube assembly 10 may include a tracheostomy tube assembly and a cricothyrotomy tube assembly. The airway tube assembly 10 may include a wire guide 18, a dilator 14, an airway tube 16, and a clip 12. The wire guide 18 may be any device over which the dilator 14 or airway tube 16 may be advanced or retracted. Examples of the wire guide 18 may include a wire, a coil, or a catheter. The wire guide 18 may be small enough to be advanced into an incision on the body of the patient.

The dilator 14 may be any device which may advanced into the incision on the body of the patient to provide a transition between the small diameter wire guide 18 and the comparatively large diameter airway tube 16. Examples of the dilator 14 may include a catheter, a cone, or any other shape capable of widening the incision. The dilator 14 may be advanced over the wire guide 18 or in some cases, may be advanced without a wire guide 18.

The airway tube 16 may be any device which, when inserted into the airway of a patient, is capable of providing an alternative airway for the patient. Examples of the airway tube 16 may include a tracheostomy tube and a cricothyrotomy tube. The dilator 14 may be placed within a lumen (64 in FIG. 8) of the airway tube 16 when the airway tube 16 is advanced into the airway of the patient. Once the airway tube 16 is sufficiently seated into the airway of the patient, the dilator 14 may be removed.

The clip 12 may be any device which contacts the airway tube 16 and the dilator 14 to restrain the movement of the airway tube 16 and the dilator 14 with respect to each other. Examples of the clip 12 may include an attachment, a seal, or a brace. The clip 12 may be coupled to the dilator 14 and the airway tube 16 while the airway tube assembly 10 is being advanced into the airway of the patient. The clip 12 may be subsequently removed to facilitate retraction of the dilator 14 from the airway tube 16.

FIG. 2 illustrates a cross-sectional side view of an example of the clip 12. The clip 12 may include a body 24, a proximal element 20 and a distal element 22. The body 24 may be any structure on the clip 12 which is adapted to extend along a portion of the length of the airway tube 16 and dilator 14. Examples of the body 24 may include a strut, a tube, or a rod. The body 24 may have a length between 66 mm and 98 mm, including any value within range or endpoints, but preferably may be 82 mm. The body 24 may be made of any material, such as metal or plastic, which is rigid enough to maintain the position of the proximal element 20 and the distal element 22 with respect to the dilator 14 and the airway tube 16 when the airway tube assembly 10 is under normal operational stresses. However, the body 24 may be made of a material which is flexible enough to allow the clip 12 to be separated from the airway tube 16 and dilator 14 by the hand force of an operator.

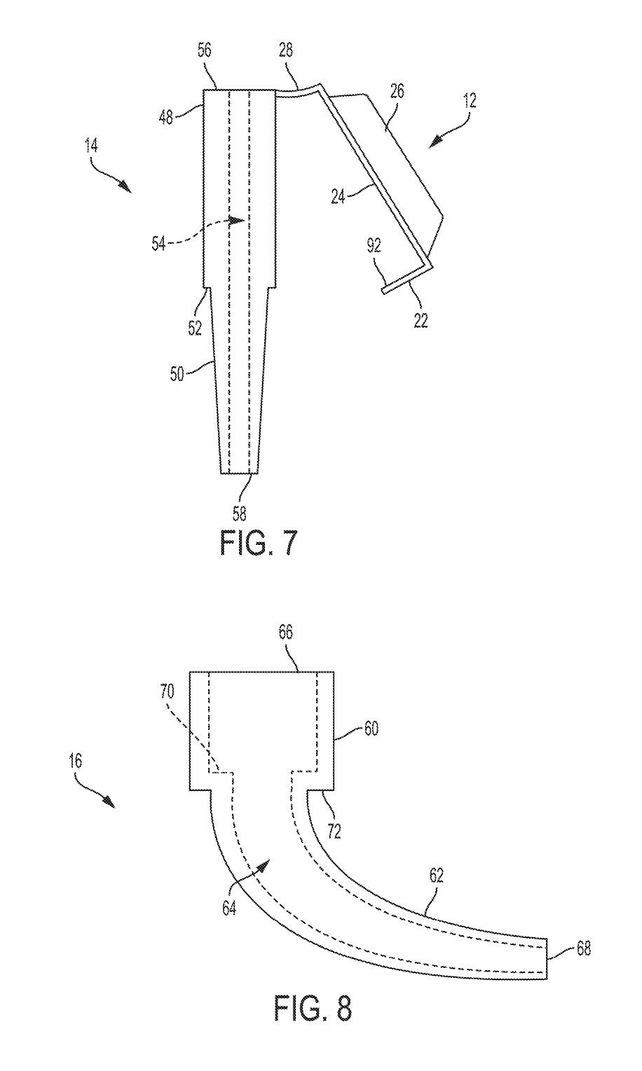

The proximal element 20 may be any structure which projects outwardly from a proximal end of the body 24, which is adapted to extend over a proximal end of the airway tube assembly 10, and which is adapted to restrict distal movement of the dilator 14 relative to the clip 12. Examples of the proximal element 20 may include a cap, a cage, or a plate. In some embodiments, the proximal element 20 may be adapted to extend over a proximal end (56 in FIG. 7) of the dilator 14 and thereby restrict the proximal movement of the dilator 14. The proximal element 20 may have a length between 6.4 mm and 9.6 mm, but preferably may be 8.0 mm. The proximal element may also have a diameter of between 8.8 mm and 13.2 mm, but preferably may be 11.0 mm. In other embodiments, the proximal element 20 may extend over the airway tube 16 and may include an interior surface 42 which projects into the airway tube 16 to contact the proximal end 56 of the dilator 14 and restrict the proximal movement of the dilator 14. The proximal element 20 may be separated from the body 24 by a proximal arm 28 which extends outwardly from the proximal end of the body 24 and which is coupled to the proximal element 20.

The interior surface 42 may be any surface which is adapted to contact the proximal end 56 of the dilator 14. Examples of the interior surface 42 may include a groove, a planar surface, or a dome. In some embodiments, the proximal element 20 may be a cap, including a lip 34. The lip 34 may be any structure of the proximal element 20 which is adapted to encircle a portion of the airway tube assembly 10 and which restricts the lateral motion of the proximal element 20 relative to the airway tube assembly 10. Examples of the lip 34 may include a plurality of downward facing projections, a semi-circular ring, or a cylindrical structure. The lip 34 may define a proximal opening 38 to receive the airway tube assembly 10. The proximal opening 38 may be sized to receive either the proximal end of the dilator 14 or the proximal end of the airway tube 16 to contact the interior surface 42 within the proximal opening 38. The proximal opening 38 may have a diameter of between 7.2 mm and 10.8 mm, but preferably may be 9.0 mm. The interior surface 42 may also define an aperture 40. The aperture 40 may be sized to receive the wire guide 18 and may be positioned at the center of the proximal element 20. The aperture 40 may be smaller than the proximal opening 38 and may be smaller in diameter than the proximal end of the dilator 14 or the proximal end of the airway tube 16.

The distal element 22 may be any structure which projects outwardly from the distal end of the body 24, which is adapted to be coupled to the airway tube assembly 10, and which is adapted to restrict proximal movement of the airway tube 16 relative to the clip 12. Examples of the distal element 22 may include pincers, an arm, or a clamp. In some embodiments, the distal element 22 may be coupled to a distal arm 30 which projects outwardly from the distal end of the body 24. The distal element 22 may have a diameter of between 8.8 mm and 13.2 mm, but preferably may be 11.0 mm. The distal element 22 may have a length between 2.4 mm and 3.6 mm, but preferably may be 3.0 mm. The distal element 22 may also include a gripping element 32 which encircles and defines a distal lumen 36. The distal lumen 36 may be sized and adapted to receive a component of the airway tube assembly 10, such as the airway tube 16. The distal lumen 36 may have a diameter of between 5.6 mm and 8.4 mm, but preferably may be 7.0 mm. In some embodiments, when coupled to the airway tube 16, the gripping element 32 may restrict the lateral movement of the airway tube 16 relative to the clip 12. However, when sufficient force is applied between the clip 12 and the airway tube 16, the gripping element 32 may release the airway tube 16, allowing relative lateral motion between the airway tube 16 and the clip 12. The distal element 22 and the proximal element 20 may be aligned with one another such that the proximal lumen 38 and the aperture 40 overlap with the distal lumen 36. The distal element 22 may also include an engagement surface 92 adapted to engage with the outer surface of the airway tube 16. The engagement surface 92 may be proximally facing or may be inwardly facing.

The body 24 of the clip 12 may also include a tab 26 projecting outwardly from the body 24. The tab 26 may be any structure that is adaptable to be held by the user. Examples of the tab 26 may include a handle, a flange, or a textured surface. The tab 26 may project outwardly from the body 24 in a direction opposing the direction of projection of the proximal element 20 and the distal element 22. The tab 26 may be angled such that a user may grip the tab 26 to exert a lateral force on the clip 12.

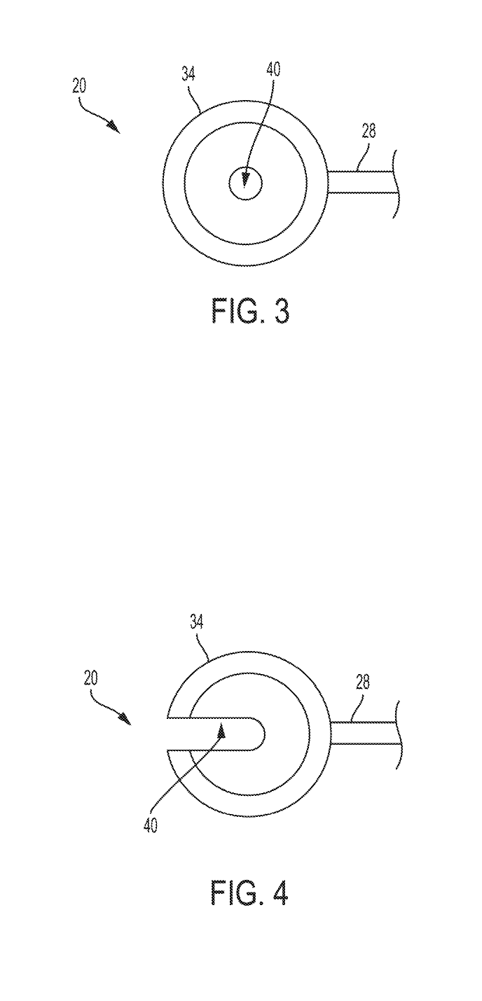

FIG. 3 illustrates an example of an embodiment of the proximal element 20. In some embodiments, the aperture 40 may be substantially circular in shape to accommodate the wire guide 18. In such embodiments, the clip 12 may be advanced to the airway tube assembly 10 or retracted away from the airway tube assembly 10 over the wire guide 18.

FIG. 4 illustrates an alternative embodiment of the proximal element 20. In some embodiments, the aperture 40 may resemble a slot extending from the center of the proximal element 20 to an outer surface of the lip 34. In such an embodiment, the clip 12 may be coupled to the airway tube assembly 10 without advancing the clip 12 over the wire guide 18, as the wire guide 18 enters the aperture 40 through the side of the lip 34. The clip 12 may be then moved distally such that the proximal end of the airway tube assembly 10 contacts the interior surface 42 of the proximal element 20.

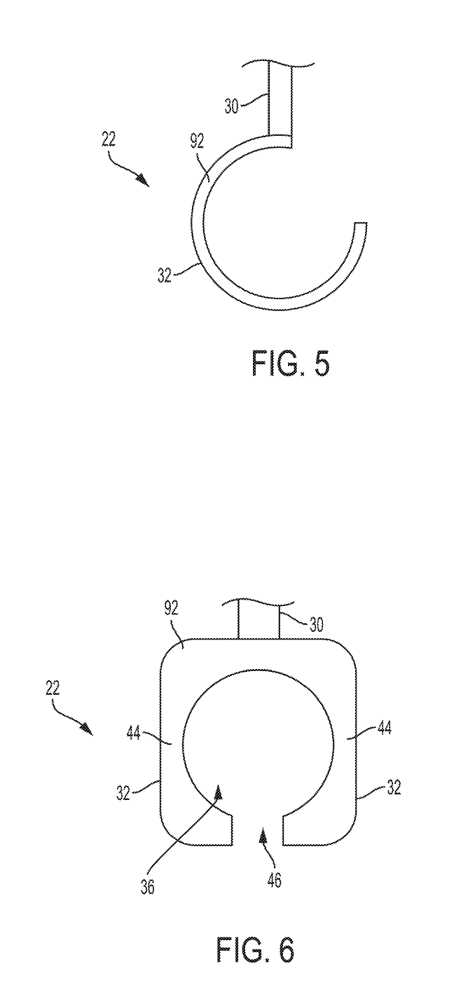

FIG. 5 illustrates an example of an embodiment of the distal element 22. In some embodiments, the gripping element 32 may be a hook which is adapted to encircle more than half of the circumference of the airway tube 16. In such an embodiment, the gripping element 32 may restrict the lateral movement of the airway tube 16 relative to the clip 12. However, such an embodiment may also allow the griping element 32 to be uncoupled from the airway tube 16 by moving the distal element to unhook the distal element 22 from the airway tube 16. The gripping element 32 may be sufficiently rigid to restrict lateral motion of the airway tube assembly 10 relative to the clip 12. The gripping element 32 may also be sufficiently flexible to release the airway tube assembly 10 when a predetermined force is applied to the clip 12.

FIG. 6 illustrates an alternative embodiment of the distal element 22. In some embodiments, the distal element 22 may include two opposing gripping elements 32, each gripping element 32 being adapted to encircle a portion of the circumference of the airway tube assembly 10. The opposing gripping elements 32 may define a gap 46 on an opposing side of the distal element 22 as the distal arm 30. The gap 46 may have a width which is less than a diameter of the airway tube assembly 10, allowing the gripping elements 32 to restrict the lateral motion of the airway tube assembly 10 relative to the clip. The opposing gripping elements 32 may also be movable to release the airway tube assembly 10 when a sufficient force is applied. For example, in some embodiments, the opposing gripping elements 32 may be expandable on a hinge to release the airway tube assembly 10. In such an embodiment, the opposing gripping elements 32 may be biased to grip the airway tube assembly 10 by a spring, which released when the spring is compressed. In other embodiments, opposing gripping elements 32 may have flexible portions 44 which are able to plastically bend to receive or release the airway tube assembly 10. In some embodiments, the flexible portions 44 of the gripping elements 32 may be the thinnest portion of the gripping element 32.

FIG. 7 illustrates an embodiment of the dilator 14 which includes an attached clip 12. The clip 12 may be coupled to the dilator 14 by the proximal arm 28. Therefore, in this embodiment, the proximal element 20 of the clip 12 may be the proximal end 56 of the dilator 14. In such embodiments, the position of the dilator 14 with respect to the clip 12 may be fixed. As shown in FIG. 7, the proximal arm 28 of the clip may be flexible to allow the clip 12 bend when being attached to the airway tube 16.

In some embodiments, the dilator 14 may include a proximal portion 48 and a distal portion 50. The proximal portion 48 may be adapted to extend out of the proximal end (66 in FIG. 8) of the airway tube 16 and may have a uniform diameter. The distal portion 50 of the dilator 14 may be tapered and may be adapted to extend out of the distal end (68 in FIG. 8) of the airway tube 16 to ease the movement of the airway tube assembly 10 into the airway of the patient. The proximal portion 48 and distal portion 50 of the dilator 14 may be separated by a distal-facing surface 52. The distal-facing surface 52 may be adapted to engage with a proximal-facing surface (70 in FIG. 8) to define the furthest distal position of the dilator 14 with respect to the airway tube 16. The dilator 14 may also include a lumen 54 extending from the proximal end 56 to a distal end 58 of the dilator which is adapted to receive the wire guide 18.

FIG. 8 illustrates an example of the airway tube 16 including a lumen 64 extending from the proximal end 66 to the distal end 68 of the airway tube 16. As discussed above, the lumen 64 may be adapted to receive the dilator 14. The airway tube 16 may include a distal portion 62 and a proximal portion 60. The proximal portion 60 may be adapted to remain outside the patient and may have a uniform diameter. The distal portion 62 may be tapered to ease insertion of the airway tube 16 into the airway of the patient. The diameter of the proximal portion 60 may be greater than the diameter of the distal portion 62.

In some embodiments, the proximal portion 60 may be separated from the distal portion 62 by an external engagement surface 72. The external engagement surface may be any surface which is adapted to engage with the engagement surface 92 of the distal element 22 of the clip 12. Examples of the external engagement surface 72 may include a partial conical portion, a slot in the outer surface of the airway tube 16, a groove in the outer surface of the airway tube 16, or a step between the diameter of the proximal portion 60 and the distal portion 62 of the airway tube 16. The external engagement surface 72 may be adapted to engage with the engagement surface 92 of the distal element 22 of the clip 12 to restrict the distal movement of the airway tube 16 relative to the clip 12. Therefore, when the distal-facing surface 52 of the dilator is engaged with the proximal-facing surface 70 of the airway tube 16, the interior surface 42 of the proximal element 20 of the clip 12 is engaged with the proximal end 56 of the dilator 14, and the external engagement surface 72 of the airway tube 16 is engaged with the engagement surface 92 of the distal element 22 of the clip 12, the positions of the dilator 14, the airway tube 16, and the clip 12 may all be fixed relative to one another. This stable configuration may ease insertion of the airway tube assembly 10 into the airway of the patient.

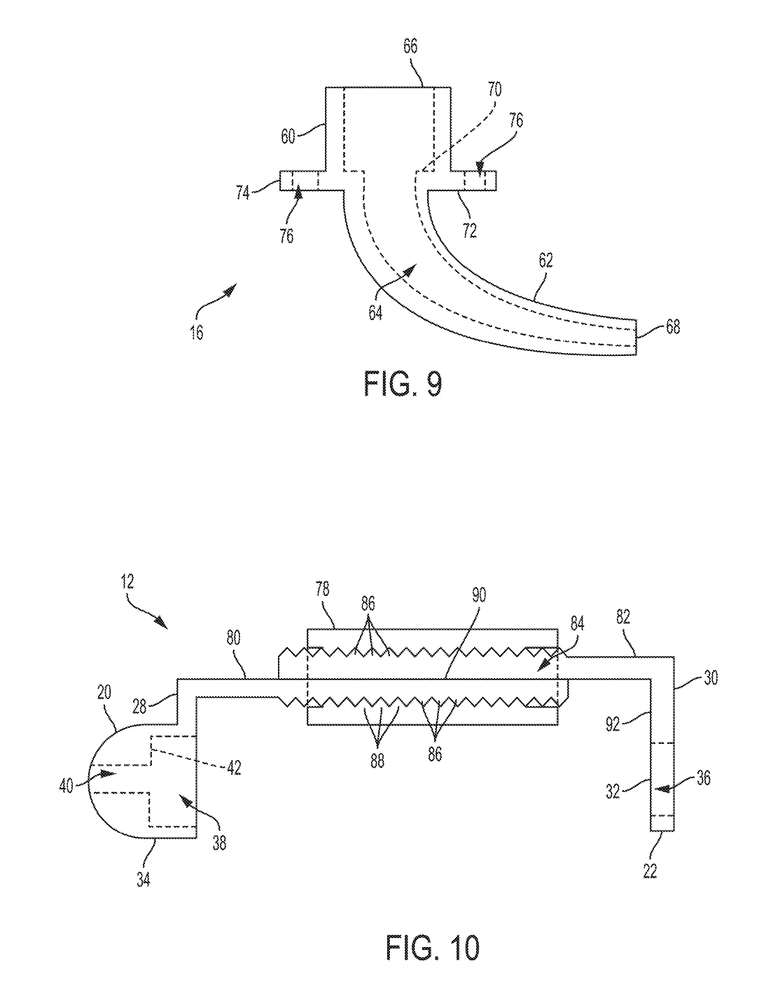

FIG. 9 illustrates an alternative embodiment of the airway tube 16. In some embodiments, the external engagement surface 72 may take the form of a flange extending radially from the outer surface of the airway tube 16. This embodiment may provide a larger external engagement surface 72 for the engagement surface 92 of the distal element 22 of the clip 12 to engage with. The flange may also include an opening passing through the flange to allow the flange to be sutured to the patient once the airway tube 16 is inserted into the airway of the patient.

FIG. 10 illustrates an alternative embodiment of the clip 12. Where the sizes of the dilator 14, the airway tube 16, and the airway tube assemblies are not precisely known, the length of the clip 12 may need to be adjustable to accommodate a variety of configurations. Therefore, in some embodiments, the clip 12 may have an adjustable length by operation of an adjustment mechanism 78. The adjustment mechanism 78 may be any device which allows the adjustment of the length between the interior surface 42 of the proximal element 20 and the engagement surface 92 of the distal element 22. Examples of the adjustment mechanism 78 may include a ratchet mechanism, a stretchable elastic body, or a telescoping body. As shown in FIG. 10, the adjustment mechanism 78 may also include a turnscrew device including a proximal body portion 80 and a distal body portion 82 extending through the adjustment mechanism 78. The adjustment mechanism 78 may have a lumen 84 which includes internally-facing threads 88. The proximal body portion 80 and the distal body portion 82 may be situated within the lumen 84 of the adjustment mechanism 78 and may contact one another along a sliding surface 90. The proximal body portion 80 and the distal body portion 82 may also include externally-facing teeth or threads 86 which engage with the internally-facing threads 88 of the adjustment mechanism 78. When the adjustment mechanism 78 is rotated about an axis of the body 24 (defined by the sliding surface 90), the relative positions of the proximal body portion 80 and the distal body portion 82 may change, increasing or decreasing the length between the interior surface 42 of the proximal element 20 and the engagement surface 92 of the distal element 22.

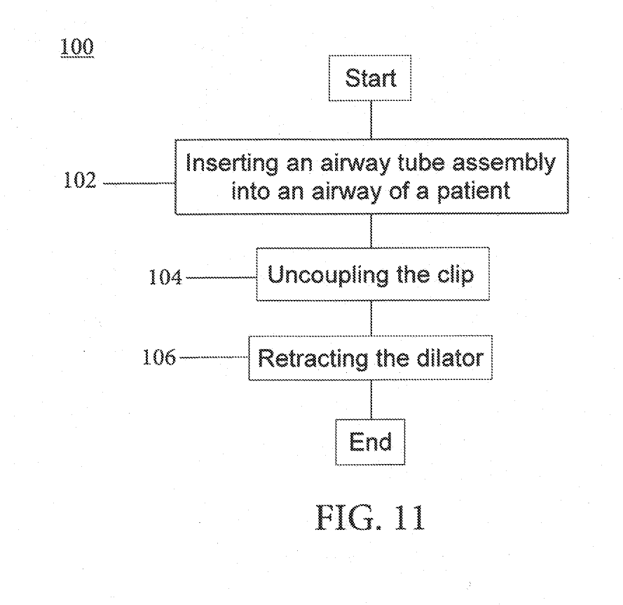

FIG. 11 illustrates a flow diagram of an example of a method of inserting the airway tube 16 into the airway of the patient (100). The steps may include additional, different, or fewer operations than illustrated in FIG. 11. The steps may be executed in a different order than illustrated in FIG. 11.

The airway tube assembly 10 is inserted into the patient (102), the airway tube assembly 10 including the airway tube 16, the dilator 14, and the clip 12. The airway tube assembly 10 may be advanced into the patient over the wire guide 18. After the airway tube assembly 10 has been fully inserted into the airway of the patient, the clip 12 may be uncoupled from the airway tube 16 (104). After the clip 12 has been uncoupled from the airway tube 16, the dilator 14 may be retracted from the lumen 64 of the airway tube 16 (106).

In some embodiments, the method (100) may further include coupling the clip 12 to the airway tube 16 and to the proximal end 56 of the dilator 14 before advancing the airway tube assembly 10 into the airway of the patient. In other embodiments, the method (100) may also include advancing the clip 12 distally over the wire guide 18 prior to coupling the clip 12 to the airway tube 16. In other embodiments, the method (100) may also include adjusting the length of the clip 12 from the proximal element 20 to the distal element 22 such that, when coupled to the airway tube assembly 10, the clip 12 restricts relative movement between the airway tube 16 and the dilator 14.

The logic illustrated in the flow diagrams may include additional, different, or fewer operations than illustrated. The operations illustrated may be performed in an order different than illustrated.

To clarify the use of and to hereby provide notice to the public, the phrases “at least one of <A>, <B>, . . . and <N>” or “at least one of <A>, <B>, <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed.

While the preferred embodiments of the disclosed have been described, it should be understood that the invention is not so limited and modifications may be made without departing from the disclosure. The scope of the disclosure is defined by the appended claims, and all devices that come within the meaning of the claims, either literally or by equivalence, are intended to be embraced therein.

What is claimed is:

1. A clip for use with an airway tube assembly comprising a dilator and an airway tube, the clip comprising:
a body extending from a proximal end to a distal end;
a proximal element projecting outwardly from the proximal end of the body, the proximal element adapted to extend over a proximal end of the airway tube assembly and to restrict proximal movement of the dilator relative to the clip; and
a distal element projecting outwardly from the distal end of the body, the distal element adapted to be coupled to the airway tube assembly and to restrict distal movement of the airway tube relative to the clip.

2. The clip of claim 1, wherein the proximal element is a cap comprising a lip adapted to encircle a portion of the airway tube assembly to secure the clip to the proximal end of the airway tube assembly.

3. The clip of claim 2, wherein the cap further comprises an interior surface adapted to engage with the proximal end of the airway tube assembly, wherein the interior surface of the cap is adapted to restrict proximal movement of the airway tube assembly relative to the clip.

4. The clip of claim 3, wherein the interior surface defines an opening at the center of the cap, wherein the opening is sized to receive a wire guide.

5. The clip of claim 4, wherein the opening is a slot extending from the center of a cap to an outer surface of the lip.

6. The clip of claim 1, wherein the distal element comprises an arm adapted to encircle at least a portion of the airway tube assembly.

7. The clip of claim 6, wherein the distal element comprises a pair of opposing arms, each arm adapted to encircle at least a portion of the airway tube assembly and in combination, restrict lateral motion of the airway tube assembly.

8. The clip of claim 7, wherein the pair of opposing arms are sufficiently rigid to restrict lateral motion of the airway tube assembly but sufficiently flexible to release the airway tube assembly when a predetermined lateral force is applied to the clip.

9. The clip of claim 1, wherein the body of the clip comprises a tab projecting outwardly from the body in a direction opposing the proximal element and the distal element.

10. An airway access system comprising:
an airway tube comprising an outer surface and an inner surface defining a lumen;
a dilator adapted to be placed within the lumen of the airway tube, wherein the dilator comprising a proximal end; and
a clip comprising a body extending from a proximal end to a distal end, the body comprising a proximal element projecting outwardly from the proximal end of the body, the proximal element adapted to be positioned at the proximal end of the dilator and to restrict proximal movement of the dilator relative to the clip, and a distal element projecting outwardly from the distal end of the body, the distal element adapted to be coupled to the airway tube and to restrict distal movement of the airway tube relative to the clip.

11. The airway access system of claim 10, wherein the proximal element of the clip is integrally coupled to the dilator.

12. The airway access system of claim 10, wherein the outer surface of the airway tube further comprises a distally-facing surface adapted to engage with the distal element of the clip.

13. The airway access system of claim 12, wherein the distally-facing surface of the outer surface of the airway tube comprises a flange projecting outwardly from the outer surface of the airway tube.

14. The airway access system of claim 12, wherein the distally-facing surface of the outer surface of the airway tube comprises an indentation in the outer surface.

15. The airway access system of claim 10, wherein the body has a length between the proximal element and the distal element which is adjustable.

16. The airway access system of claim 10, wherein the inner surface of the airway tube is adapted to receive the dilator and restrict the distal movement of the dilator relative to the airway tube.

17. A method of inserting an airway tube into a patient, comprising:
inserting an airway tube assembly into the patient, the airway tube assembly comprising the airway tube having an inner surface defining a lumen, a dilator disposed within the lumen of the airway tube, wherein the dilator having a proximal end, and a clip comprising a body extending from a proximal end to a distal end, the body comprising a proximal element projecting outwardly from the proximal end of the body, wherein the proximal element is coupled to the proximal end of the dilator to restrict proximal movement of the dilator relative to the clip, and a distal element projecting outwardly from the distal end of the body, wherein the distal element is coupled to the airway tube to restrict distal movement of the airway tube relative to the clip;
uncoupling the clip from the airway tube; and
retracting the dilator from the lumen of the airway tube.

18. The method of claim 17, further comprising coupling the clip to the airway tube and to the proximal end of the dilator before inserting the airway tube assembly into the airway of the patient.

19. The method of claim 18, further comprising adjusting a length of the clip from the proximal element to the distal element such that the clip restricts relative movement between the airway tube and the dilator.

20. The method of claim 17, further comprising advancing the clip distally over a wire guide prior to coupling the clip to the airway tube and to the proximal end of the dilator.
